SEQUENCE LISTING (8 sequences)

SEQ ID NO: 1 - protein, 10 aa, artificial sequence
  Cleavage substrate
KGGLQRALEI

SEQ ID NO: 2 - protein, 8 aa, artificial sequence
  Cleavage substrate
GLQRALEI

SEQ ID NO: 3 - protein, 8 aa, artificial sequence
  Cleavage substrate
GGLQRALE

SEQ ID NO: 4 - protein, 9 aa, artificial sequence
  Cleavage substrate
GGLQRALEI

SEQ ID NO: 5 - protein, 10 aa, artificial sequence
  Cleavage substrate
ESLGRKIQIQ

SEQ ID NO: 6 - protein, 8 aa, artificial sequence
  Cleavage substrate
SLGRKIQI

SEQ ID NO: 7 - protein, 8 aa, artificial sequence
  Cleavage substrate
ESLGRKIQ

SEQ ID NO: 8 - protein, 9 aa, artificial sequence
  Cleavage substrate
SLGRKIQIQ

(19) United States
(12) Patent Application Publication
(10) Pub. No.: US 2005/0037949 A1
(43) Pub. Date: Feb. 17, 2005

(54) Mannose binding lectin and uses thereof

(76) Inventors: Grace O'Brien, Victoria (AU); Robert Neil Pike, Victoria (AU); Melinda Margaret Dean, Queensland (AU); Robyn Myra Minchinton, Queensland (AU); Teresa Marion Martinelli, Victoria (AU)

Correspondence Address: SCULLY SCOTT MURPHY & PRESSER, PC, 400 GARDEN CITY PLAZA, GARDEN CITY, NY, 11530, US

(21) Appl. No.: 10/484,373

(22) PCT Filed: Apr. 24, 2003
(86) PCT No.: PCT/AU03/00489

(30) Foreign Application Priority Data
Apr. 24, 2002 (AU) PS 1961
Dec. 13, 2002 (AU) 2002953324

(51) Int. Cl. A61K 38/16

(52) U.S. Cl. 514/8; 530/396

(57) ABSTRACT

The present inventors have shown that MASP-depleted MBL is able to recruit MASPs from plasma and successfully activate complement cascade. Furthermore, it has been discovered that MBL purified as a complex has limited ability to activate the complement cascade when compared to MASP-depleted MBL. Accordingly, the present invention provides a pharmaceutical composition comprising an isolated non-recombinant mannose binding lectin (MBL) substantially free from activated MBL associated serine proteases (MASPs) together with a pharmaceutically acceptable carrier or diluent. Also provided is a method of treating a subject in need of MBL comprising administering to the subject an effective amount of a pharmaceutical composition of the invention.

### FIELD OF THE INVENTION

The present invention relates to purified mannose binding lectin (MBL), substantially free from MBL associated serine proteases (MASPs) and its use in therapy.

### BACKGROUND TO THE INVENTION

Mannose binding lectin (MBL), sometimes referred to as mannan binding lectin or mannose binding protein, is a liver derived C-type serum lectin with structural homology to complement component C1q. MBL can activate complement via the lectin and classical pathways, and can interact with specific C1q-like receptors on the surface of phagocytes, thus playing an important role in first-line host defence.

MBL is a member of the collectin family of proteins that are characterised by the presence of both a collagenous region and a globular lectin domain. The structural unit of MBL is a 96 kDa collagen triple helix of three 32 kDa subunits, each with a carbohydrate-recognition domain. The helix is stabilised by disulphide bonds between N-terminal cysteines. MBL oligomerizes as multiples of this 96 kDa unit and the native protein is commonly found as trimers to hexamers ranging from 270 kDa to approximately 650 kDa. MBL full functionality is only obtained when it is in its higher oligomeric forms. There is evidence that MBL must at least be tetrameric to enable effective complement activation. This oligomeric structure allows MBL multiple ligand binding sites and mimics the multiple binding characteristics of IgM.

MBL binds many different sugars, but binds most avidly to mannose and N-acetylglucosamine. These sugars are prevalent on the cell walls of many pathogens such as yeast, gram negative enteric bacteria, gram positive bacteria, mycobacteria, some viruses, and certain parasites. As most of the MBL sugar targets are not expressed at high densities on the surface of mammalian cells, MBL has the ability to distinguish self from non-self. MBL thus serves as a pattern recognition molecule in the first-line of host defence, a central part of the so-called innate immune system (Turner, 1996).

Central in the efficient and effective complement activation function of MBL is its close association in vivo with at least two pro-enzymes called MBL associated serine proteases 1, 2 and 3 (MASP1, MASP2 and MASP-3). These single polypeptides of 93 kDa, 76 kDa and 105 kDa, respectively become activated when MBL binds its ligand and promote efficient complement activation via the lectin pathway (Turner, 1996). It has been demonstrated that MASP2 is essential for complement activation and this enzyme alone is capable of initiating the complement cascade without the presence of either MASP1, or the recently described MASP3. MASP2 is thus the critical enzyme associating with MBL to promote activation of the complement cascade.

The MBL gene (MBL2) is located on chromosome 10 at 10q11.2-q21 and contains four exons. A number of mutations in MBL2 that have an impact on the expression of functional protein have been described. Single nucleotide substitutions in codons 52, 54 and 57 of exon 1 of the MBL2 gene are believed to disrupt the assembly of MBL subunits into the basic trimeric structural unit.

In addition, at least two polymorphisms have been described in the promoter region (at positions −550 and −221 respectively) that alter the level of expression of individual MBL sub-units. The frequency of mutations in the MBL gene varies among ethnic groups. For example the codon 54 variant occurs with a frequency of 15% in Caucasians while the codon 57 variant is seen exclusively in Africans. The practical significance of the common occurrence of both the gene mutations and the promoter polymorphisms is that MBL deficiency is relatively common in the general population. The serum level of MBL in individuals homozygous for the wild-type gene ranges from 1 to 5 μg/mL while those individuals homozygous for MBL2 mutations have levels of 5 to 25 ng/mL and heterozygous individuals have levels approximately ⅛^{th} normal, but there is considerable observed variation in levels.

A number of lines of evidence suggest that MBL deficiency has clinically important consequences.

A childhood syndrome of recurrent infections, failure to thrive and chronic diarrhoea was first linked to an in vitro opsonic defect of plasma in 1968. It was subsequently confirmed that this syndrome was associated with low MBL levels in 10 children aged from 15 mths to 9 yrs. The importance of MBL2 deficiency as a risk factor for childhood infection was confirmed in a consecutive series of 345 children admitted to hospital with infection. The prevalence of MBL2 gene mutations in children with infection was twice that in those without infection and the increased susceptibility was seen in both heterozygote and homozygote individuals. Infections seen ranged from chest infections and otitis media through to life threatening meningococcaemia.

The association of MBL deficiency with meningococcal disease in children has been confirmed in a large study of 266 cases. 7.7% of the hospital based cases were homozygous for MBL polymorphisms in comparison to 1.5% of the control group giving an odds ratio of 6.5. It was concluded that the genetic variants of MBL may account for a third of all cases.

These data in the paediatric population have led to the hypothesis that the major role of MBL is to provide protection during the so called “window of vulnerability” that occurs after maternal antibodies are lost and before the maturation of an infants own antibody repertoire (6 mths to 18 mths).

The recent findings that MBL genotypic variants are associated with an early age of onset of presentation of common variable immunodeficiency and acute lymphoblastic leukaemia adds weight to the hypothesis that MBL mediated host defence takes on greater importance when other components of the immune system are immature or impaired.

Common genetic variations in the MBL gene have recently been associated with increased disease severity and risk of infection with Burkholderia cepacia in 149 cystic fibrosis (CF) patients. MBL variant alleles were also associated with poor prognosis and early death—predicted age of survival was reduced by 8 years in variant allele carriers when compared with normal homozygotes in the CF population.

There is increasing evidence of the clinical importance of MBL deficiency in adults. Four adult patients with “severe and unusual” infections (including recurrent skin infections, Cryptosporidiosis, Meningococal meningitis with recurrent herpes simplex and oesophageal candidiasis, and Klebsiella pneumonia) were shown to have MBL2 mutations involving either codons 52 or 54.

In 228 adult patients suspected of having non-HIV-related immunodeficiency, the frequency of heterozygosity for MBL2 mutations was the same as a control population. However, there was a significant increase in homozygous MBL2 mutations amongst those with presumed immunodeficiency (8.3% vs 0.8%). Data have also been presented showing that the risk of HIV infection is greater and the rate of progression of AIDS is faster in men homozygous for MBL polymorphisms.

In patients in whom the adaptive immune response has been compromised by chemotherapeutic regimens, the effect of MBL structural gene mutations and low levels of circulating MBL has been clearly associated with increased incidence of infection and severity of infection. Adults receiving chemotherapy for haematological malignancies with MBL levels below 0.5 μg/ml had significantly increased incidence and severity of infection. Donor and recipient MBL genotype were found to be important in influencing the risk of infection in adults following allogeneic stem cell transplantation. Amongst 100 children undergoing chemotherapy, those with structural MBL gene mutations had twice as many days of febrile neutropenia as those with wild type MBL genes and four of these were admitted to ICU with infection. MBL levels less than 1 μg/ml were thought to be critical in this study.

In one of the few prospective, community based studies yet performed 252 children were examined (Koch et al., 2001). It was discovered that MBL deficiency as strongly linked (twice the risk) to acute respiratory infection in children aged 6 to 17 months. MBL deficiency had less impact in those aged 0 to 5 months and had no impact on acute respiratory infection in those aged 18 to 23 months.

MBL-MASP complex has been purified routinely on a laboratory scale since 1980. MBL-MASP complex purification has been performed by affinity chromatography in various forms. The ligand is usually yeast mannan (Anderson et al., 1992; Holmskov et al., 1993). One or two cycles through the column are performed with the first elution with high salt or EDTA (Koppel et al., 1994; Anderson et al., 1992; Holmskov et al., 1993) and the final elution with mannose (Koppel et al., 1994; Anderson et al., 1992; Matsushita et al., 1992; Holmskov et al., 1993).

Human MBL-MASP complex has also been purified from a waste fraction produced during the fractionation of plasma proteins, on a laboratory scale (Kilpatrick, 2000), and under GMP conditions at the Statens Serum Institut (Valdimarsson et al., 1998). Scottish Cohn fraction m is a waste product of IgG production by plasma fractionation. Cryosupernatant produced from plasma is precipitated with 21% ethanol. The precipitate from this step is called fraction I+II+III. A further precipitation with 8% ethanol produces fraction I+m from which MBL-MASP complex can be affinity captured using an Emphaze-mannan column. Elution of MBL-MASP complex was achieved with first EDTA, then mannose solutions. The yield of MBL-MASP complex from this procedure is quoted as 10 mg/kg of fraction I+III paste; a specific activity seven fold greater than pooled plasma (Kilpatrick, 2000). In this way, highly pure MBL-MASP complex (300-600 μg/litre plasma) can be recovered with simple mannose elution.

An alternative purification technique is discussed in WO99/64453 which discloses a chromatographic purification step using a non-conjugated polysaccharide matrix.

### SUMMARY OF THE INVENTION

The present inventors have discovered that MASP-depleted MBL compositions are superior at activating the complement cascade when compared to MBL purified in complex with its associated MASPs. Consequently, the present inventors have found that for the purpose of formulating a safe, effective therapeutic product for administration to subjects, MASPs, or at least activated MASPs should be removed from the MBL during, or prior to, or after purification of the MBL-MASP complex

Accordingly, in a first aspect, the present invention provides a pharmaceutical composition comprising isolated non-recombinant mannose binding lectin (MBL) substantially free from activated MBL associated serine proteases (MASPs) together with a pharmaceutically acceptable carrier or diluent.

Preferably the composition is substantially free of MASPs, whether activated or not. Typically, the MBL is human MBL.

The present inventors have found that MASP-depleted MBL is able to recruit MASPs from plasma to produce a functional complex that can successfully activate the complement cascade. In contrast, it appears that purified MBL-MASP complex has a limited capacity to recruit proenzyme (or fresh) MASP. This is probably due to the presence in the purified complex of activated MASP attached to the binding sites on MBL as a result of activation during the purification process (e.g. being activated upon binding to mannan columns), the activated MASP being difficult to displace. By contrast, proenzyme MASPs can be freshly recruited to available binding sites on purified MASP-depleted MBL. This also restores the regulation component of the MBL-MASP complex, making it a safer, more effective therapeutic product.

In a preferred embodiment, the MBL is obtained by a method comprising:
(i) providing a complex of non-recombinant MBL and one or more MASPs;
(ii) incubating the complex in a suitable buffer to dissociate the MBL from the one or more MASPs; and
(iii) separating the MBL from the one or more MASPs.

Preferably, the buffer in step (ii) is an EDTA/acetate buffer at a pH of from 4.0 to 5.0.

Furthermore, it is preferred that the buffer comprises NaCl. Preferably, the buffer has an NaCl concentration of at least 0.5 M. More preferably, the buffer has an NaCl concentration of about 1 M.

Preferably step (iii) includes a chromatographic method and/or filtration. In a further preferred embodiment, the chromatographic method is selected from the group consisting of: size exclusion chromatography and ion exchange chromatography.

In a second aspect, the present invention also provides a method of producing a pharmaceutical composition, the method comprising:
(i) providing a complex of non-recombinant MBL and one or more MASPs;
(ii) dissociating the MBL from at least some of the one or more MASPs;
(iii) separating the MBL from at least some of the one or more MASPs; and
(iv) admixing the resulting MBL from step (iii) with a pharmaceutically acceptable carrier or diluent.

Preferably, step (ii) involves incubating the complex in a suitable buffer.

Preferably, the buffer is an EDTA/acetate buffer at a pH of from 4.0 to 5.0.

Furthermore, it is preferred that the buffer comprises NaCl. Preferably, the buffer has an NaCl concentration of at least 0.5 M. More preferably, the buffer has an NaCl concentration of about 1 M.

Preferably step (iii) includes a chromatographic method and/or filtration. In a further preferred embodiment, the chromatographic method is selected from the group consisting of: size exclusion chromatography and ion exchange chromatography.

In a preferred embodiment step (i) comprises providing a side fraction from plasma fraction processes. Preferably step (i) further comprises separating complexes of non-recombinant MBL and one or more MASPs from other plasma proteins present in the side fraction from plasma fraction processes by mannan affinity chromatography.

The present invention also provides a pharmaceutical composition obtained by the method of the second aspect of the invention. Preferably, the composition is substantially free of activated MASPs

In another aspect, the present invention provides a method of treating or preventing a disease in a subject, the method comprising administering to the subject an effective amount of a pharmaceutical composition of the invention.

The disease can be any condition, the treatment or prevention of which would be aided by the subject being administered with purified MASP-depleted MBL. Examples of suitable recipients of the method include, but are not limited to, bone marrow allograft recipients, subjects with cystic fibrosis, subjects with an immunodeficiency, subjects with acute lymphoblastic leukaemia, subjects with community acquired or nosocomial septicaemia, subjects with or susceptible to an infection by a pathogen, low birthweight and/or premature infants. Typically, the subject has an MBL deficiency.

The present invention also provides a composition comprising isolated non-recombinant MBL, said composition being substantially free of MASPs, for use prophylactically or in therapy.

The present invention further provides the use of a composition comprising isolated non-recombinant MBL, said composition being substantially free of MASPs, in the manufacture of a medicament for use in administering to a subject in need of said composition.

Examples of suitable recipients include, but are not limited to, bone marrow allograft recipients, subjects with cystic fibrosis, subjects with an immunodeficiency, subjects with acute lymphoblastic leukaemia, subjects with community acquired or nosocomial septicaemia, subjects with or susceptible to an infection by a pathogen, low birthweight and/or premature infants. Typically, the subject has an MBL deficiency.

The present inventors have also devised cleavage substrates, and assays for the use thereof, for determining the levels of MASP activity in a sample. Such assays can be used for monitoring MBL purification procedures described herein, or for any other purpose where it is desirable to analyse MASP activity.

Thus, in a further aspect the present invention provides a peptide of formula X-R1-Arg-R2-Y wherein R1-Arg-R2 is a peptide consisting of 6 or more contiguous amino acids derived from the MASP cleavage site of a complement protein; X is NH₂, a blocking group or a detectable label; and Y is COOH or a detectable label, provided that when X is NH₂ or a blocking group, Y is not COOH and when Y is COOH, X is not NH₂ or a blocking group.

Preferably, the complement protein is C4.

Preferably, the C4 protein is human C4 and the cleavage site comprises Arg756.

In a further preferred embodiment, X is a quencher molecule and Y is a fluorescent label, or vice-versa, such that a fluorescent signal is obtained when the substrate is cleaved.

In a further aspect, the present invention provides for the use of a peptide of the invention in a method of determining the presence of MASP activity in a sample.

In yet another aspect, the present invention provides a method of determining the presence of MASP activity in a sample which method comprises contacting the sample with a peptide according to the invention and determining whether said peptide has been cleaved.

In a further aspect, the present invention provides a method of producing a pharmaceutical composition of the invention which method comprises:
(i) providing a complex of non-recombinant MBL and one or more MASPs;
(ii) incubating the complex in a suitable buffer to dissociate the MBL from the one or more MASPs;
(iii) separating the MBL from the one or more MASPs;
(iv) screening the MBL obtained from (iii) for MASP activity using a method of the invention; and
(v) admixing the resulting purified MBL with a pharmaceutically acceptable carrier or diluent.

### BRIEF DESCRIPTION OF THE ACCOMPANYING DRAWINGS

FIG. 1: Plots of MBL levels against C4 deposition for MASP-depleted MBL and MBL-MASP complex demonstrating superior in vitro MASP2 recruitment and complement activation by MASP-depleted fractions. C4 results at excess are plotted at value of 1.0.

FIG. 2: Plots of MBL levels against C4 deposition for MASP-depleted MBL and MBL-MASP complex demonstrating superior in vitro MASP2 recruitment and complement activation by MASP-depleted fractions. C4 results at excess are plotted at value of 1.0.

### DETAILED DESCRIPTION OF THE INVENTION

Unless defined otherwise, all technical and scientific terms used herein have the same meaning as commonly understood by one of ordinary skill in the art (e.g. in cell culture, molecular genetics, nucleic acid chemistry, hybridization techniques and biochemistry). Standard techniques are used for molecular, genetic immunology and biochemical methods (see generally, Sambrook et al, Molecular Cloning: A Laboratory Manual, 3rd ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), which are incorporated herein by reference) and chemical methods.

A. Purification of MBL Substantially Free of MASPs.

Purified MASP-depleted MBL according to the present invention is obtained from non-recombinant sources—i.e. by purification from animal or human biological material such as plasma. However, the MBL present in such material is complexed with MASPs and therefore the purification of MBL substantially free of activated MASPs according to the invention requires the separation of MBL from those MASPs.

The purification process typically involves two major steps—the purification of MBL-MASP complex from other biological material and the dissociation of MBL-MASP complexes to obtain purified substantially MASP-depleted MBL. These two steps can occur in any order or even at the same time. However, typically a pre-purification step is performed to remove at least some biological material, such as non-MBL-MASP plasma proteins, prior to the dissociation step, and to enrich for MBL-MASP complexes since MBL often constitutes less then 0.05% of the total protein content of plasma. Thus the biological material, such as blood plasma, is typically treated to obtain a partially purified composition comprising MBL-MASP complex.

One starting point for MBL purification is blood, blood plasma, liver and liver cell cultures. However, MASP-depleted MBL can also be purified from plasma-derived products or by-products. Preferably, the source of MASP-depleted MBL is from a side fraction from plasma fractionation processes. Examples include, but are not limited to, precipitates or supernatants from precipitation processes, or filtrates, or side fractions from ion exchange chromatography, or side fractions from affinity chromatography, or fractions from other processes which are not used to produce other plasma based products. As the skilled addressee would be aware, there are many different known plasma fractionation processes. However, the skilled addressee can readily screen for MBL, for example performing mannan binding, MBL antigen or C4 deposition assays and/or affinity chromatography purification of MBL on different fractions as described herein, to determine which fractions of a given plasma fractionation process comprises MBL.

An example of a side fraction from plasma fractionation processes as a source of MASP-depleted MBL is crude plasma protein fractions from industrial scale ethanol fractionation procedures, such as Cohn fractions II and/or m. These fractions, which include MBL-MASP complex containing paste derived from Cohn supernatant I (referred to herein as “euglobulin paste”) are usually discarded and therefore they are economically advantageous as a starting material. This is because blood is a valuable and rare resource and it is therefore desirable to maximise the use of such side fractions.

The source of MASP-depleted MBL may be from animals or humans. However, it is preferred to purify MASP-depleted MBL from human sources.

Where plasma/plasma by-products are used, they are generally treated to enrich for plasma proteins. Typically, plasma proteins are obtained from the plasma or plasma-derived products etc. by a precipitation process. Plasma proteins can be precipitated from plasma or plasma by-products using a variety of suitable agents known in the art including various molecular weight forms of poly(ethylene glycol), ethanol and ammonium sulphate.

Further optional steps include filtration, such as depth filtration, and delipidation.

For example, a euglobulin paste may be obtained as follows: thawed freshly frozen plasma is treated with water for injection (WFI) and cold ethanol at a temperature of below 5° C. The resulting precipitate is then separated by centrifugation. Typically, the supernatant is delipidated to adsorb lipoproteins and clarified by filtration. The supernatant is then diafiltered using ultrafiltration membrane with a nominal molecular weight cut off of not less than 10 000 Daltons to lower the conductivity. The pH of the diafiltered supernatant is lowered to promote euglobulin precipitation and the clarified supernatant is recovered by filtration. Euglobulin paste is collected during this process.

MBL-MASP complexes are typically extracted from other plasma proteins by affinity purification, which separates the MBL (most of which is complexed to MASP) from other plasma proteins. Generally, the affinity capture ligand is a sugar. Examples include, but are not limited to, mannan and N-acetylglucosamine. In a preferred embodiment, the affinity capture ligand mannan e.g. mannan-Sepharose or mannan-agarose. Where MBL-MASP complexes are present in a precipitate, such as euglobulin paste, the precipitated proteins are re-solubilised prior to loading onto the affinity resin. A suitable solubilisation buffer is Tris/NaCl/CaCl₂ buffer. For example, euglobulin paste can be solubilised in a Tris/NaCl/CaCl₂ buffer for 1 hour at room temperature.

Non-solubilised material is generally removed by centrifugation and/or filtration. Solubilised plasma protein precipitate is loaded onto the affinity resin and the resin washed prior to elution of MBL-MASP complexes with a calcium ion chelating agent, such as EDTA.

An alternative method for purifying MBL-MASP complexes is described in WO99/64453 which uses a polysaccharide matrix, without any conjugated carbohydrate ligands such as mannan. Since MBL can bind directly to the polysaccharide matrix, purification can be effected in a similar manner to mannan affinity resins but without the need to prepare a conjugated affinity resin.

MBL-MASP complex containing solutions, obtained as described above or by other suitable means, are then treated to dissociate the MBL complex i.e. to dissociate MASPs from the MBL. For example, this can be achieved by incubating the MBL complex in a suitable buffer comprising sodium acetate buffer (pH 4.0-5.0) and EDTA In addition, the buffer may further comprise NaCl. Suitable concentrations of NaCl for the dissociation of MBL-MASP complexes can readily be determined using techniques known in the art. In one embodiment, the buffer has an NaCl concentration of at least 0.5 M. More preferably, the buffer has an NaCl concentration of about 1 M.

Purified MASP-depleted MBL is then obtained by a suitable purification step to separate the MBL from MASPs. Separation is typically on the basis of size/molecular weight, e.g. size exclusion chromatography, filtration and/or electrophoresis, or on the basis of charge eg. ion exchange chromatography, but other suitable means may be employed. For example, Sephacryl S-300 size exclusion chromatography or filtration may be used. MBL containing fractions are collected and typically concentrated.

Throughout the purification process, it may be desirable at one or more stages to include a concentration step to increase the concentration of MASP-depleted MBL and/or MBL-MASP complexes. This is achieved by the affinity purification step but in addition, one or more ultrafiltration steps may be included. Preferably, the membranes used for ultrafiltration have a molecular weight cutoff of from 10,000 Da to 100,000 Da. It is generally desirable to maintain the MBL and/or MASP complexes in a compatible buffer during the concentration steps.

It is preferred during the MASP-depleted MBL purification steps to include one or more viral inactivation steps since the MASP-depleted MBL is obtained from animal/human biological products. Viral inactivation techniques are known in the art and typically comprise contacting the MBL with a virus-inactivating agent such as a detergent/solvent combination. Suitable detergents are described in U.S. Pat. No. 4,314,997 and U.S. Pat. No. 4,315,991 and include Triton X-100 and Tween 80. Suitable solvents include di- and trialkylphosphates such as tri(n-butyl) phosphate.

By an “isolated” non-recombinant MBL we mean non-recombinant MBL which is at least partially separated from molecules with which it is associated or linked in its native state. Preferably, the isolated non-recombinant MBL is at least 50% free, preferably at least 75% free, and more preferably at least 90% free from other components with which it is naturally associated.

The present inventors have shown that the removal of MASPs bound to non-recombinant MBL enhances the ability of MBL to activate the complement pathway. As the skilled addressee would be aware, the removal of any activated MASPs bound to MBL during purification will enhance the activity of the MBL component. Naturally, the more bound activated MASPs that are removed the more active the MBL component will be. Accordingly, the present invention extends to any pharmaceutical composition which has higher ratios of non-recombinant MBL to activated MASPs when compared to the staring source (for example plasma).

In one aspect the invention provides a composition comprising purified non-recombinant MBL substantially free of activated MASPs, particularly activated MASP-2. In this context, the term “substantially free” means that a composition comprising 5 μg of isolated non-recombinant MBL provides a C4 deposition assay result of greater than about 0.3 U/μl. Preferably, the C4 deposition assay result is greater than about 0.5 U/μl, more preferably greater than about 0.75 U/μl, more preferably greater than about 1 U/μl, more preferably greater than about 1.25 U/μl, and even more preferably greater than about 1.5 U/μl. Suitable C4 deposition assays are known in the art and described herein.

In another aspect the present invention provides methods of purifying non-recombinant MBL substantially free of activated MASP. In this context, and in one embodiment, the term “substantially free” is determined as outlined above using a C4 deposition assay. Alternatively, the term “substantially free” can be determined when comparing the MASP activity of the starting material, namely before any method step dissociating MBL-MASP complexes, to the MASP activity of the at least partially purified MBL product. In a preferred embodiment, the MASP activity is reduced by at least 75%, more preferably at least 80%, more preferably at least 85%, more preferably at least 90%, more preferably at least 95%, more preferably at least 97%, and even more preferably at least 99%. MASP activity assays are known in the art and include those described herein.

The term “activated MASP” means that the MASP is not in its pro-enzyme form but is in its active form, due to proteolysis. Activated MASPs can be distinguished from pro-enzyme by size—the pro-enzyme has a molecular weight of about 70 to 110 kDa whereas the activated enzyme consists of a heavy chain of about 50 to 65 kDa and a light chain of about 30 to 40% Da. These can be resolved by gel electrophoresis under reducing conditions followed by visualisation and/or immunodetection (e.g. Western blotting).

However, in a preferred embodiment, the MBL is substantially free of MASPs, whether activated or not as calculated relative to MBL-MASP complex.

MBL functions more effectively as an oligomer due to increased avidity. Consequently, it is preferred that the purified MASP-depleted MBL of the invention remains in its native oligomeric state. Preferably at least 50% of the purified MBL is present as oligomers, more preferably as tetramers or higher order oligomers.

B. Pharmaceutical Compositions

Purified MASP-depleted MBL according to the invention may be combined with various components to produce MASP-depleted MBL compositions. The compositions typically comprise a pharmaceutically acceptable carrier or diluent to produce a pharmaceutical composition (which may be for human or animal use) to produce a pharmaceutical composition of the invention. The phrase “pharmaceutically acceptable” refers to molecular entities and compositions that do not produce an allergic, toxic, or otherwise adverse reaction when administered to an animal, particularly a mammal, and more particularly a human. Pharmaceutically acceptable media or carriers include any and all solvents, dispersion media, coatings, antibacterial and antifungal agents, stabilizers, isotonic and absorption delaying agents and the like. The use of such media and agents for pharmaceutical active substances is well known in the art.

Suitable carriers and diluents include isotonic saline solutions, for example phosphate-buffered saline. The carrier can also be a solvent or dispersion medium containing, for example, water, ethanol, polyol (for example, glycerol, propylene glycol, and liquid polyethylene glycol, and the like), suitable mixtures thereof and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Examples of suitable stabilizers include, but are not limited to, pharmaceutical grades of a monosaccharide, a disaccharide, sucrose, lactose, trehalose, mannitol, sorbitol, inositol, dextran and the like; plasma protein products other than MBL or MASP such as albumin; amino acids; and polyols (for example, polyethylglycol) and the like.

The composition may be in any suitable form such as a liquid or a solid. Solid compositions may be obtained using any technique known in the art including spray-drying, freeze-drying, spray-freeze drying, air-drying, vacuum-assisted drying, fluid bed drying and the like.

The composition of the invention may be administered by direct injection. The composition may be formulated for various routes of administration including parenteral, intramuscular and intravenous administration. Other delivery systems can include time-release, delayed release or sustained release delivery systems. Such systems can avoid repeated administrations of the MASP-depleted MBL compositions of the invention, increasing convenience to the subject and the physician. Many types of delayed release delivery systems are available and known to those of ordinary skill in the art. They include polymer-based systems such as polylactic and polyglycolic acid, polyanhydrides and polycaprolactone; nonpolymer systems include lipids such as sterols, and particularly cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, pump-based hardware delivery systems can be used, some of which are adapted for implantation.

A long-term sustained release implant also may be used. Long-term release, as used herein, means that the implant is constructed and arranged to deliver therapeutic levels of purified MBL for at least 30 days, and preferably 60 days. Long-term sustained release implants are well known to those of ordinary skill in the art.

Typically, MBL protein may be administered at a dose of from 0.001 to 100 mg/kg body weight, preferably from 0.01 to 10 mg/kg, more preferably from 0.05 to 1 mg/kg body weight.

For example, a suitable initial dose for an MBL deficient adult is 6 mg MBL in 100 ml saline, given as an infusion, with follow up doses of about 6 mg twice weekly as required.

The routes of administration and dosages described are intended only as a guide since a skilled practitioner will be able to determine readily the optimum route of administration and dosage for any particular patient and condition.

Compositions of the present invention may be co-administered with compositions comprising unactivated purified MASPs. Such MASPs suitable for co-administration may be obtained recombinantly using techniques known in the art. The nucleotide sequence of human MASP-1 is available as GenBank Accession No. NM_{—}001879. The nucleotide sequence of human MASP-2 is available as GenBank Accession AH010229. The nucleotide sequence of human MASP-3 is available as GenBank Accession AF284421.

C. Assays for MASP Activity

It is desirable to assay the MBL compositions of the invention to confirm that they are substantially free of MASP. The presence of MASPs can be measured using a variety of techniques including immunological methods (e.g. BLISA). In addition, MASP activity can be determined using assays based on cleavage of labelled substrates—such as labelled peptides derived from the C-terminus of the products of MASP cleavage of complement proteins—C2, C3, C4 or C5.

Further, we have developed a highly sensitive assay method for active MASP based on the use of substrates derived from the MASP cleavage site on the C4 protein. These substrates differ from those disclosed in U.S. Pat. No. 6,235,494 because they contain amino acid sequences from both sides of the cleavage site of the uncleaved complement protein whereas the substrates of U.S. Pat. No. 6,235,494 do not contain any amino acids residue C-terminal of the arginine cleavage site. The inclusion of the additional amino acids, such that the arginine is flanked by amino acids, provides additional specificity and reliability.

Accordingly, the present invention provides a peptide of formula X-R1-Arg-R2-Y wherein R1-Arg-R2 is a peptide consisting of 6 or more contiguous amino acids derived from the MASP cleavage site of a complement protein; X is NH₂, a blocking group or a detectable label; and Y is COOH or a detectable label provided that when X is NH₂, or a blocking group, Y is not COOH and when Y is COOH, X is not NH₂ or a blocking group.

Preferably R1 and/or R2 comprise at least three amino acids, preferably at least four amino acids. Preferably R1-Arg-R2 comprises fewer than 10 amino acids. More preferably R1-Arg-R2 consists of 7 or 8 amino acids.

The complement protein cleavage site from which R1-Arg-R2 is derived is preferably the MASP cleavage site of a C2, C3, C4 or C5 protein, such as Arg756 of human C4 (Accession No. P01028).

Examples of peptides of the invention include, but are not limited to:

| | |
|---|---|
| X-Lys-Gly-Gly-Leu-Gln-Arg-Ala-Leu- | (SEQ ID NO:1) |
| Glu-Ile-Y | |
| X-Gly-Leu-Gln-Arg-Ala-Leu-Glu-Ile-Y | (SEQ ID NO:2) |
| X-Gly-Gly-Leu-Gln-Arg-Ala-Leu-Glu-Y | (SEQ ID NO:3) |
| X-Gly-Gly-Leu-Gln-Arg-Ala-Leu-Glu- | (SEQ ID NO:4) |
| Ile-Y | |
| X-Glu-Ser-Leu-Gly-Arg-Lys-Ile-Gln- | (SEQ ID NO:5) |
| Ile-Gln-Y | |
| X-Ser-Leu-Gly-Arg-Lys-Ile-Gln-Ile-Y | (SEQ ID NO:6) |
| X-Glu-Ser-Leu-Gly-Arg-Lys-Ile-Gln-Y | (SEQ ID NO:7) |
| X-Ser-Leu-Gly-Arg-Lys-Ile-Gln-Ile- | (SEQ ID NO:8) |
| Gln-Y | |

Derivatives of naturally occurring complement protein cleavage site sequences may also be used. The term “derivatives” means that minor substitutions, insertions and deletions may be made to the naturally occurring complement protein cleavage site sequences, other than the arginine residue, provided that the resulting sequences can be cleaved by one or more MASPs and that at least two, preferably at least three or at least four, amino acid residues are present C-terminal of the arginine cleavage site.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-changed | D E |
| | | K R |
| AROMATIC | | H F W Y |

Substitutions may also include the use of non-naturally occurring amino acid analogues.

Considering the disclosure herein, the skilled addressee could readily screen derivates (either comprising naturally and/or non-naturally occurring amino acids) of labelled known MASP cleavage substrates to determine suitable derivates which could be used in the assays of the present invention.

At least one of X or Y are detectable labels that permit detection of cleavage of the substrate. Any suitable detectable labels may be used, such a radiolabels, colorimetric, bioluminescent, chromogenic or fluorescent labels. However, in a preferred embodiment, one of the labels is a fluorescent label. In a highly preferred embodiment, X is a fluorescent label and Y is a quencher molecule, or vice-versa. In this way, provided that X and Y are within a certain distance of each other (e.g. 8 or less amino acids apart) in the uncleaved substrate, no fluorescence signal will be obtained. However on cleavage of the substrate by a MASP, the quencher molecule will be separated from the fluorescent label and fluorescent signal will be obtained.

Examples of quencher molecules include, but are not limited to, dinitrophenyl ethylenediamine (EDDnp) and Lys(Dnp). Examples of fluorescent labels include, but are not limited to, 7-amino-4-methylcoumarin (AMC) and aminobenzoic acid (Abz). Examples of colourimetric molecules include, but are not limited to, para-nitroaniline.

Peptides of the invention are typically made by synthetic means using techniques well known to skilled persons such as solid phase synthesis. Various techniques for chemically synthesising peptides are reviewed by Borgia and Fields (2000) and described in detail in the references contained therein.

The assay systems herein may be provided in kit form that is useful for determining activated MASP levels in a sample. The kits may include a substrate contained in a suitable container or linked to a solid support, such as a microtiter plate or other suitable support, or contained in the wells of a microtiter plate. Kits may also include instructions for performing the assays.

The kits will optionally include other reagents for performing the assays, including controls, trypsin, Futhan or other serine protease inhibitor, buffers, such as PBS, stop solutions, and other such reagents. The kits may also include suitable ancillary supplies, such as microtiteir plates, vials, labeled ligand or labeled anti-ligand, calibrator solutions, controls, wash solutions, solid-phase supports and the like.

The peptides of the invention can be used to assay for MASP activity in a sample, such as a sample containing MASP-depleted MBL purified as described above. Samples may also include biological samples, such as blood samples, from patients, including patients suspected of having an MBL deficiency.

Accordingly, the present invention provides a method of determining the presence of MASP activity in a sample which method comprises contacting the sample with a peptide of the invention and determining whether said peptide has been cleaved.

The method for detection of proteolytic activity, i.e. cleavage of the substrate, will vary depending on the type of label. Detection can, for example, be based on quantitative or qualitative measurements.

For quantitative measurements, typically the signal emitted by the label is measured from the beginning of the reaction and the results used to obtain an initial rate. Substrates consisting only of the residues N-terminal to the cleavage site of C4 show normal Michaelis-Menten kinetics for their cleavage by both C1s and the MBL-MASP complex. This means that the dependence of the initial velocity for the cleavage reaction on substrate concentration can be described by a rectangular hyperbola and the constants Kₘ (Michaelis constant which equates to the affinity between enzyme and substrate) and Vₘₐₓ (maximal velocity of the reaction) can be derived from a non-linear regression fit of the data.

Substrates which incorporate amino acid residues both N- and C-terminal to the cleavage point of C4, do not show Michaelis-Menten kinetics for the cleavage of the substrate by C1s and MBL-MASPs, however. Instead, the dependence of initial velocity for the cleavage on substrate concentration is best described by a sigmoidal curve. This indicates that the enzyme is displaying allosteric behaviour or positive co-operativity in the cleavage of P₄-P₄′ substrates. Non linear regression fitting of the curve in this case yields three different constants: Vₘₐₓ (again, the maximal velocity of the interaction), K_{0.5} (or the substrate concentration at half Vₘₐₓ, which indicates the affinity between enzyme and substrate) and the Hill constant (h, indicating the degree of positive co-operativity).

It has been reported previously that C1 inhibitor (C1INH) binds to the MASPs at a ratio of 1:1. Thus a preparation of C1INH of known active concentration can be used to titrate the amount of active enzyme in MASP preparations. This can be carried out using C1s as a positive control. This will then allow the calculation of k_{cat} for the interaction between MASPs and fluorometric substrates. Once activity (fluorescence) is plotted against C1INH concentration, the active enzyme concentration of the MASP preparation can be determined as the point at which the line intercepts the x-axis.

The K_{0.5} and Vₘₐₓ values for an enzyme substrate reaction can then be determined using allosteric kinetics. Knowledge of the active enzyme concentration then allows calculation of the k_{cat} constant for the enzyme substrate reaction using the following equation:
k_{cat}=Vₘₐₓ/[active enzyme]

Where Vₘₐₓ is the maximal velocity of the enzyme-substrate reaction and [active enzyme] is the molar amount of enzyme present in the preparation that is capable of cleaving substrate.

Once the k_{cat} value has been determined, MASP preparations can be assayed at substrate concentrations twice the K_{0.5} value, yielding a velocity that is nominally equivalent to Vₘₐₓ. The k_{cat} and Vₘₐₓ values can then be substituted into the equation: k_{cat}=Vₘₐₓ/[active enzyme]. Rearrangement of the equation will yield an estimate of the active enzyme concentration in the sample.

D. Therapeutic Uses

The pharmaceutical compositions of the present invention may be used to treat subjects in need of MBL.

As used herein, an “effective amount” means an amount sufficient to at least increase the ability of the subjects immune system to opsonise pathogens and induce the complement cascade in response to the pathogen.

Subjects include bone marrow allograft recipients, subjects with cystic fibrosis, subjects with an immunodeficiency, subjects with acute lymphoblastic leukaemia, subjects with community acquired or nosocomial septicaemia, subjects with or susceptible to an infection by a pathogen, low birthweight and/or premature infants. Typically, the subject has an MBL deficiency, such as congenial MBL deficiency.

As used herein, an MBL deficiency is where the subjects MBL levels are below 500 ng/ml and/or the subjects C4 deposition assay result is less than 0.3U/ul. In particular individuals having an MBL level below 400 ng/ml will benefit from the methods of the invention, such as individuals having an MBL level below 300 ng/ml, or such as individuals having an MBL level below 250 ng/ml, or such as individuals having an MBL level below 200 ng/ml.

The pathogen may be any organism which comprises a molecule to which MBL binds resulting in activation of a complement pathway. Such pathogens may be yeast, gram negative enteric bacteria, gram positive bacteria, mycobacteria, some viruses, and certain parasites. More specific examples of such pathogens include, but are not limited to, those selected from the group consisting of: Parasites such as Cryptospridium parvum and Plasmodium falciparum; Fungi such as Cryptococcus sp. including Cryptococcus neoformans, Candida albican and Aspergillus fumigatus; and Bacteria such as beta haeniolytic streptococcus group A, Bifidobacterium bifidum, Actinomyces israelli, Proprionibacterium acnes, Bacteroides sp., Escherichia coli, Eubacterium sp., Fusobacterium sp., Veillonella sp., Haemophilus influenzae, Neisseria gonorrhoeae, Neisseria meningitidis, Staphylococcus aureus, Salmonella enterica, Burkholderia cepacia and Klebsiella pneumoniae.

Particular indications include: Neurology: Chronic inflammatory demyelinating polyneuropathy (CIDP), Multifocal motoric neuropathy, Multiple sclerosis, Myasthenia Gravis, Eaton-Lambert's syndrome, Opticus Neuritis, Epilepsy; Gynaecology: Abortus habitualis, Primary antiphospholipid syndrome; Rheumatology: Rheumatoid arthritis, Systemic lupus erythematosus, Systemic scleroderma, Vasculitis, Wegner's granulomatosis, Sjogren's syndrome, Juvenile rheumatoid arthritis; Haematology: Autoimmune neutropenia, Autoimmune haemolytic anaemia, Neutropenia; Gastrointestinal: Crohn's disease, Colitis ulcerous, Coeliac disease; Others: Asthma, Septic shock syndrome, Chronic fatigue syndrome, Psoriasis, Toxic shock syndrome, Diabetes, Sinuitis, Dilated cardiomyopathy, Endocarditis, Atherosclerosis, Adults with AIDS and bacterial infections, Primary hypo/agammaglobulinaemia including common variable immunodeficiency, Wiskot-Aldrich syndrome and severe combined immunodeficiency (SCID), Secondary hypo/agammaglobulinaemia in patients with chronic lymphatic leukaemia (CLL) and multiple myeloma, Children with AIDS and bacterial infections, Acute and chronic idiopathic thrombocytopenic purpura (ITP), Allogenic bone marrow transplantation (BMT), Kawasaki's disease, and Guillan-Barre's syndrome.

It has been shown that a deficiency in MBL predisposes infants to acute lymphoblastic leukaemia. Consequently, the methods of the invention may also be used prophylactically to prevent disorders caused by/associated with MBL deficiency, such as acute lymphoblastic leukaemia. Thus, subjects also include those at risk of developing any of the above disorders, as appropriate, due to an MBL deficiency, such as MBL-deficient infants at increased risk of developing acute lymphoblastic leukaemia.

The present invention will now be described further with reference to the following Examples, which are illustrative only and non-limiting.

### EXAMPLES

### Example 1

### Purification of MASP-Depleted MBL

Fresh frozen plasma was softened and thawed at temperatures below 5° C. and the cryoprecipitate separated from the cryosupernatant by continuous flow centrifugation. Cold ethanol was added to the cryosupernatant to a final concentration of 8% (v/v). The precipitate formed was separated from the supernatant by centrifugation or filtration at −2° C.±1° C. The supernatant was treated to adsorb lipoproteins and clarified by filtration. Delipidated supernatant was diafiltered using ultrafiltration membrane with nominal molecular weight cut off of not less than 10 000 Daltons to lower the conductivity. The pH of the delipidated diafiltered supernatant was lowered to promote euglobulin precipitation and the clarified supernatant recovered by filtration. The euglobulin paste collected during this process was further purified to extract MBL-MASP complex.

The purification process was carried out at ambient temperature. Euglobulin paste was solubilised in a 20 mM Tris/100 mM NaCl/15 mM CaCl₂ buffer for 1 hour at room temperature. Non-solubilised material was removed by centrifugation. Affinity chromatography was employed to separate the MBL-MASP complex from other plasma proteins. Solubilised euglobulin paste was loaded onto a mannan-agarose column. The column was washed with a Tris/NaCl/CaCl₂/Tween 20 buffer before the MBL-MASP complex was eluted with 10 mM EDTA.

The eluate was then incubated in 0.1 M sodium acetate buffer (pH 5.0) containing EDTA to dissociate MASPs from the MBL molecules. The material was then applied to a sephacryl S-300 size exclusion column and fractions analysed by SDS page. Results showed separation of MBL from other protein components. Fractions were analysed for MASP activity in the substrate assays as described in this document. Seven fractions contained MBL with low levels, or near-depleted, of MASP activity. Total protein concentration of each fraction was in the range of 10-90 μg/mL.

### Example 2

### Assays to Confirm Absence of MASPs

Pooled MBL fractions from Example 1 are tested to confirm that the MBL is substantially free of MASPs.

Substrate Design

Substrates were designed for MASPs based on the amino acids surrounding the cleavage site (⁷⁵⁶R) of the natural substrate, C4 protein. These substrates are used to determine the activity of the MASPs in the MBL purified material.

The present inventors have found that the inclusion of additional amino acid such that the arginine is flanked by amino acids, provides additional specificity and reliability (Table 1).

TABLE 1
Kinetic constants for the proteolytic activity of MASPs in purified MBL-
MASP complex on synthetic substrates based on the P₄-P₁ and P₄-P₄′
amino acid of complement protein C4

Affinity constant

| Substrate | Kₘ (μM) | K_{0.5} (μM) |
|---|---|---|
| C4 (P₄-P₁) | 198.0 ± 20.4 | — |
| C4 (P₄-P₄′) | — | 6.50 ± 0.32 |

The substrate (2Abz-GLQRALEI-Lys(Dnp)-NH₂) includes the four amino acids before and after the C4 cleavage site and an aminobenzoic acid (Abz) fluorescent group attached to its N-terminal end. The Abz group is quenched by Lys(Dnp), when located no more than 8 amino acids away from the Abz group. The cleavage site of the substrate is located between the Abz group and the Lys(Dnp) group, so that when the enzyme cleaves the substrate, the quenching ability of Lys(Dnp) is lost and the Abz group is able to fluoresce. The change in fluorescence can then be measured and is proportional to the proteolytic activity of the enzyme. It has been demonstrated that MASPs present in the purified MBL material cleave this substrate (K_{0.5}=6.5 I.

If proteolytic activity (i.e. a change in fluorescence) is observed when assaying the purified MBL material, this indicates that the MASPs have not been successfully removed. ELISA or immunoblot using anti-MASP antibodies is then conducted to confirm that this finding is attributed to the proteolytic activity of the MASPs and not some other protease in the MBL product. C1s, the closest homologue of the MASPs can be used as a positive control for the substrate cleavage assays.

Substrate Cleavage Assays

The substrate is diluted in fluorescent assay buffer (FAB—50 mM tris-hydroxymethylene, 150 mM NaCl, 0.2% polyethylene glycol 8000, 0.02% sodium azide, pH 7.4) so that a final concentration equal to Vₘₐₓ is achieved. The substrate and enzymes (C1s (10 μg/mL) or purified MBL material) are incubated for several minutes in a fluorescence plate reader set at 37° C. 100 μL of diluted substrate is then transferred into wells containing 100 μL diluted enzyme (C1s or purified MBL material) and the kinetics of fluorescence is measured as follows: excitation=320 nm; emission=420 nm. Each test is performed in triplicate. The amount of fluorescence is then read off the standard curve to calculate the concentration of active MASP enzymes in the purified MBL material.

### Example 3

### MASP-Depleted MBL is Capable of Recruiting MASP from Plasma and Activating the Complement Cascade

Standard Curve and Control Material for Quantitation Assays

All quantitation assays were standardised using an international, primary standard pool serum (Statens Serum Institut, Copenhagen, Denmark), containing 3.3 μg MBL/ml serum. For the sandwich ELISA and the C4 deposition assay, a standard curve was made with 1:25, 1:50, 1:75, 1:100, 1:150 and 1:200 dilutions of this serum, tested in triplicate. Standard dilutions for the mannan binding ELISA were 1:25, 1:50, 1:100, 1:150, 1:200, 1:300 and 1:400. Diluents were as detailed below. An in-house secondary control was prepared from pooled normal donor plasma and run in triplicate on each test plate, the results plotted for each run. Results of any test runs, in which values obtained for the in-house control MBL were outside +/−2SD from the previously determined mean value, led to rejection of the whole run. Run to run standard curves were overlayed to ensure a constant slope and thus provide another sensitive means of quality control.

Quantitation of MBL by Double Antibody Sandwich ELISA (“Double Antibody Assay”)

This MBL antigen detection assay was based on the original method of Garred et al. (1992) except that a commercial IgG mouse monoclonal, anti-human MBL, which targets a peptide epitope in the coliagenous neck region of the MBL structural unit, was used instead of rabbit polyclonal anti-MBL.

Briefly, flat-bottomed microtitre plates (Nunc-Immuno Maxisorp, Nalge Nunc International) were coated overnight at 4° C. with 2 μg/ml monoclonal anti-MBL (Staten Serum Institut, Copenhagen, Denmark) in fresh 50 mM carbonate-bicarbonate buffer, pH 9.6. Normal donor plasmas were tested in triplicate, diluted to 1:25 and 1:100 in 0.1 M PBS-0.05% Tween 20, pH 7.4 (IBST), which was also the wash buffer. After 90 minutes at 22° C., wells were washed 3 times and monoclonal anti-MBL biotinylated using Biotin Tag (Sigma-Aldrich Pty Ltd) was added at 1:4000, this dilution determined by chequerboard titration with pooled normal plasma. After 90 minutes at 22° C., wells were washed 3 times and ExtrAvidin peroxidase conjugate at 1:500 was added for 40 minutes at 22° C. Colour was developed with OPD tablets and diluent (Abbott Laboratories, Illinois, USA), stopped with 1N H₂SO₄ and read immediately at 490 nm in a Bio-Rad plate reader (Bio-Rad laboratories Pty Ltd., Regents Park, Australia).

Between run coefficients of variation (CV) were 8.2% at 1:25 and 12% at 1:100. Run to run standard curves were overlayed to ensure a constant slope and thus provide another sensitive means of quality control.

Quantitation of MBL by Mannan Binding ELISA (“Mannan Biniding Assay”)

This assay measures the ability of MBL to bind to mannan coated onto a polypropylene matrix, and is based on the method of Holmskov et al. (1993).

Microtitre plates (as above) were coated overnight at 4° C. with 10 μg/ml mannan (Sigma-Aldrich Pty Ltd, Castle Hill, Australia) in fresh 50 mM carbonate-bicarbonate buffer, pH 9.6. Normal donor plasmas were tested as in the sandwich ELISA but with Tris Buffered Saline with 0.05% Tween-20 (TBST) supplemented with 15 mM CaCl₂, pH 7.5 as diluent and wash buffer. All incubations were at 22° C., the MBL and antibody capture time were each 90 minutes. Incubation with ExtrAvidin peroxidase required only 30 minutes. Colour development and reading was as for the sandwich ELISA.

Between run coefficient of variation (CV) were 6.1% at 1:25 and 8.8% at 1:100. Run to run standard curves were overlayed to ensure a constant slope and thus provide another sensitive means of quality control.

The specificity of these assays for MBL was confirmed by the linear standard curves obtained with the Statens Serum Institut primary standard pool serum. We also performed limiting dilution testing in each assay with a purified mannose binding lectin prepared by mannan affinity chromatography and confirmed in the Western immunoblot. In the mannan binding assay we also were able to block binding of plasma MBL by diluting test samples in 10 mM EDTA or 0.1 M mannose solution.

Functional Complement (C4) Deposition Assay (“C4 Deposition Assay”)

Originally described for detection of deposited C3b and C3bi by Super et al. (1989) and modified by Valdimarsson et al. (1998), this assay demonstrates deposition of C4b following activation of MBL by binding with solid-phase purified mannan.

Microtitre plates (as above) were coated overnight at 4° C. with 1 μg/mL mannan (Sigma-Aldrich Pty Ltd, Castle Hill, Australia) in fresh 50 mM carbonate-bicarbonate buffer, pH 9.6. Normal donor plasmas were tested in triplicate, diluted to 1:25 in TBST with 15 mM CaCl₂, pH 7.2 which was also the wash buffer. A 1:10 dilution was also tested and interpreted only to confirm low or near absent levels of C4 deposition in donors with low amounts of MBL. After 90 minutes at 22° C., wells were washed 5 times and MBL-deficient human serum (completely deficient in MBL, obtained with informed consent) diluted 1:20 in barbital buffer, 14 mM NaCl, 10 mM sodium barbitone and 5 mM CaCl₂ was added to wells and incubated at 22° C. for 30 minutes to enable complement activation. Wells were washed 5 times and biotinylated rabbit anti-human C4 (Sigma-Aldrich Pty Ltd) biotinylated using Biotin Tag (Sigma-Aldrich Pty Ltd) which was added at 1:1500 in TBST. Following incubation at 22° C. for 90 minutes, wells were washed 5 times and 1:500 ExtrAvidin peroxidase (Sigma-Aldrich Pty Ltd) in TBST was added and incubated at 22° C. for 40 minutes. Colour development and reading was as for the quantitation assays.

1 μl of Statens Serum Institut (SSI) MBL Standard was arbitrarily assigned 1 unit of C4 deposition activity. The assay was standardised against the SSI standard. Between run CV for the assay at 1:25 was 9.4%.

Demonstration MASP-Depleted MBL can Recruit MASP and Activate the Complement Cascade

Two fractions of the purified MBL obtained in Example 1 (fractions 3 and 5 [40 μg/mL and 80 μg/mL respectively] with both fractions demonstrating MASP activity <10% of the concentration of the MBL-MASP complex—as determined using the C4 P₄-P₁ substrate), were chosen for titration and comparison with affinity purified MBL-MASP complex. Fractions demonstrating a slope/sec of less than or equal to 10% of the S300 load material (concentrated MBL-MASP complex eluate buffer exchanged in acetate buffer) were considered MASP-depleted. The positive control for MASP-2 activity was MASP containing, affinity-purified MBL complex. All fractions obtained in Example 1 were considered MASP-depleted with MASP activity measured as slope/sec less than 2.3 (range 0.5-2.3). The slope/sec for the positive control (MASP containing affinity-purified MBL complex) was 22. Both MASP-depleted fractions and the MBL-MASP complex were titrated to provide MBL protein concentrations at 1 to 100 μg/ml. Fractions were assayed in parallel in the mannan-binding and C4-deposition assays as described in this document. The result from the Mannan-binding assay was plotted against C4 activity for each fraction.

Results

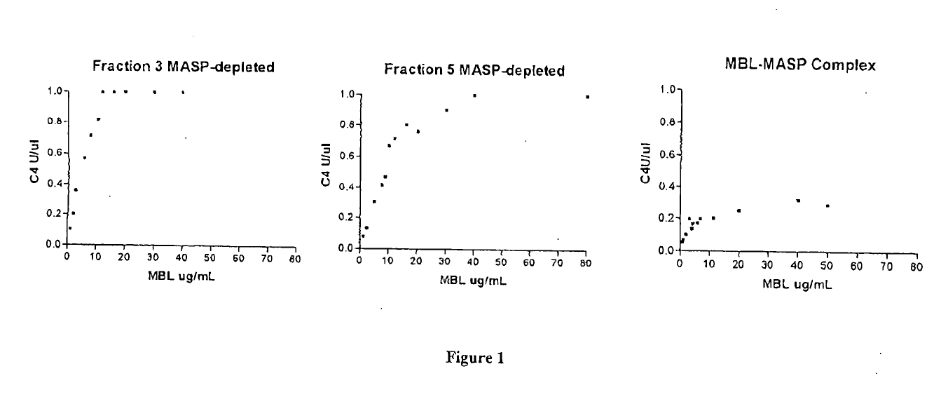

Plots of MBL levels against C4 deposition for MASP-depleted MBL and MBL-MASP complex clearly demonstrated superior in vitro MASP2 recruitment and complement activation by MASP-depleted fractions (See FIG. 1 and Table 2). Results for MBL-MASP complex began to plateau at less than 10 μg/ml MBL. A previous C4 deposition assay on the same MBL-MASP complex batch gave a C4 result of 0.27 U/μl for 25 μg/mL MBL. This is concordant with the corresponding MBL concentrations in this titration. This experiment indicates that MASPs activated by affinity purification remain docked to MBL, blocking approach of fresh MASPs when MBL binds to mannan.

TABLE 2
Titration of MASP-depleted fractions and affinity purified MBL-
MASP complex in the mannan-binding and C4-deposition assays.

| | | | | MASP-depleted | |
|---|---|---|---|---|---|
| MBL-MASP Complex | | MASP-depleted Frac. 5 | | Frac. 3 | |
| MBL ug/mL | C4 U/ul | MBL ug/mL | C4 U/ul | MBL ug/mL | C4 U/ul |
| 50 | 0.289 | 80 | xs | | |
| 40 | 0.321 | 40 | xs | 40 | xs |
| 20 | 0.25 | 30 | 0.906 | 30 | xs |
| 15 | 0.246 | 20 | 0.762 | 20 | xs |
| 11.41 | 0.204 | 16 | 0.805 | 16 | xs |
| 6.94 | 0.197 | 12 | 0.716 | 12 | xs |
| 5.95 | 0.172 | 10 | 0.671 | 10.53 | 0.818 |
| 4.42 | 0.168 | 8.79 | 0.465 | 7.97 | 0.761 |
| 4.08 | 0.138 | 7.83 | 0.417 | 5.65 | 0.568 |
| 3.3 | 0.197 | 5.11 | 0.304 | 2.57 | 0.359 |
| 2.081 | 0.103 | 2.49 | 0.137 | 2.1 | 0.202 |
| 1.032 | 0.068 | 1.32 | 0.081 | 1.02 | 0.107 |
| 0.624 | 0.05 | | | | |

Results also Plotted in FIG. 1.

### Example 4

### Confirmation of MASP Recruitment and Complement Activation by MASP-Depleted MBL

The fractions supplied in Example 3 were pooled according to total protein concentration. The first pool contained fractions 3-7 and had a total protein of 85 μg/ml. The second pool was made from fraction 8 and 9, this had a total protein concentration of 35 μg/ml. The two MASP-depleted MBL pools were again titrated in the range of 1-100 μg/ml in parallel with the MBL-MASP complex. All samples were assayed on both the Mannan-binding and C4-deposition assays in parallel. Actual MBL quantification (mannan-binding assay) results were graphed against corresponding C4 activation capability for the fraction.

Results

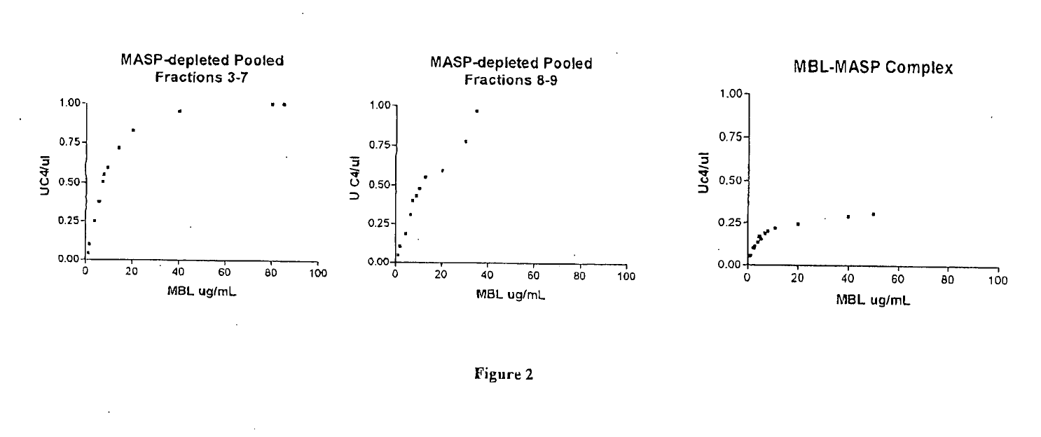

Pools of MASP-depleted MBL fractions gave reproducible results as compared to the individuals fractions analysed in Example 3. Complement activation capacity reached excess in the pooled fraction 3-7 and reached the high assay limit for fractions 8-9, whereas the MBL-MASP complex plateaued at 12 μg, and failed to substantially increase C4 deposition with increased MBL concentration. This reproduced the finding in Example 3. MASP-depleted MBL had superior ability to recruit MASP and as a result was more efficient at activating the complement cascade than the MBL-MASP complex (see FIG. 2 and Table 3).

TABLE 3
Titration of pooled MASP-depleted fractions (3-7 and 8-9) and
affinity purified MBL-MASP complex in the mannan binding and
C4 deposition assays

| MBL-MASP Complex | | MASP-depleted Pool 3-7 | | MASP Pool 8-9 | |
|---|---|---|---|---|---|
| MBL ug/mL | C4 U/ul | MBL ug/mL | C4 U/ul | MBL ug/mL | C4 U/ul |

| | | | | | |
|---|---|---|---|---|---|
| 50 | 0.308 | 85 | xs | | |
| 40 | 0.292 | 80 | xs | 35 | 0.969 |
| 20 | 0.244 | 40 | 0.954 | 30 | 0.776 |
| 10.998 | 0.22 | 20 | 0.825 | 20 | 0.595 |
| 7.967 | 0.203 | 14.045 | 0.718 | 12.718 | 0.552 |
| 6.814 | 0.19 | 9.377 | 0.596 | 10.205 | 0.48 |
| 4.681 | 0.171 | 7.796 | 0.55 | 8.995 | 0.435 |
| 5.382 | 0.159 | 7.151 | 0.506 | 7.362 | 0.403 |
| 4.011 | 0.138 | 5.617 | 0.38 | 6.506 | 0.31 |
| 2.802 | 0.114 | 3.905 | 0.25 | 4.278 | 0.189 |
| 2.237 | 0.104 | 1.677 | 0.104 | 2.032 | 0.108 |
| 1.065 | 0.057 | 1.124 | 0.043 | 1.122 | 0.049 |
| 0.471 | 0.054 | | | | |

Results also Plotted in FIG. 2

### CONCLUSIONS

The results of Examples 3 and 4 show that MASP-depleted MBL is able to recruit MASPs from plasma and successfully activate the complement cascade. Free MASPs circulate in the plasma, at levels above that of the MBL-MASP complex. Individual MASP-1 levels range from 1.48 to 12.83 μg/mL. The arithmetic mean±s.d. of MASP-1 levels in serum is 6.27+1.85 μg/mL. The serum level of MASP-1 has been found to be strongly dependent on age as is the serum MBL level. The serum level of MASP-1 has also been found to be much higher than that of MBL (1.71±1.13 μg/mL), and the major portion of human serum MASP-1 appears to exist in the circulation as a form unbound to MBL (Terai et al., 1995). MASP-2 levels are believed to be lower than MASP-1 levels. When MBL-MASP complex is disrupted by dialysis against sodium acetate buffer (pH 5.0), and then subsequently dialysed back into TBS-TEDTA buffer (pH 7.8), MBL and MASPs have been shown to be in complex. The low pH dissociation of MBL-MASP complex is reversible (Tan et al., 1996).

Furthermore these data demonstrate that MBL purified as a complex has limited ability to activate the complement cascade probably due to decreased ability to bind fresh MASPs as MASP binding sites may be blocked by MASP activated during the purification process. For instance, when MBL is purified by affinity purification on mannan columns as described previously or using a non-conjugated polysaccharide matrix as taught in WO99/64453, it co-purifies with MASPs. However, the majority of MASPs co-eluted with MBL are in their activated form, with only a fraction of MASPs remaining in their pro-enzyme (90 kDa) form due to contact with polysaccharide substrates during the affinity purification steps.

Testing of MASP-depleted MBL in the C4 deposition assay demonstrates its superior ability to recruit MASP and initiate complement activation in vitro compared with purified MBL-MASP complex. Normally, MASPs produced in the body associated with the MBL-MASP complex are only activated after specific binding of the MBL to a foreign organism. This serves as the major point of regulation for the activation of complement by the MBL pathway. Thus administering MBL containing activated MASPs eliminates this regulation mechanism.

Physiological inhibitors include C1 inhibitor (C1 INH), which forms complexes with activated MASP-1 and MASP-2. Also, C3 cleavage by MASP-1 is inhibited by C1 INH in a dose dependent manner. This is the same for C2 activation by MASP-1 and C4 & C2 activation by MASP-2. The MASPs are also inhibited by a2-macroglobulin, which has broad protease inhibitory activity (Storgaard et al 1995).

While deficiencies of an inhibitor such as C1 INH may be reasonably rare, it would also mean that individuals deficient in an inhibitor would be prone to complications following MBL-MASP complex being administered, due to inappropriate activation of the complement cascade. Thus, we consider that MBL purified with associated MASPs attached is a product with lowered efficacy and could even have potential clinical dangers.

All publications mentioned in the above specification are herein incorporated by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Where specific embodiments are described in particular sections above, the embodiments apply mutatis mutandis to other sections as appropriate.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Anderson, O. et al. (1992) Scandinavian J. Immunol. 36:131-41.
Borgia, J. A and Fields, G. B. (2000) Trends Biotech. 18:243-51.
Garred, P. et al. (1992) Eur. J. Immunogenet. 19:403-12.
Holmskov, L. et al. (1993) Glycobiology 3:147-53.
Kilpatrick, D. C. (2000) Human Reprod. 15: 94143.
Koch, A, et al. (2001) JAMA 285:1316-21.
Koppel, R et al. (1994) J. Chromatography 662:191-6.
Matsushita, M. et al. (1992) J. Exp. Med. 176:1497-502.
Storgaard, P. et al. (1995) Scan. J. Immunol. 42:373-80.
Super, M. et al. (1989) The Lancet 2:1236-9.
Tan, S. M. et al. (1996) J. Biochem. 319:329-32.
Terai, I. et al. (1995) Int. Immunol. 110:317-23.
Turner, M. W. (1996) Immunol. Today 17:532-40.
Valdimarsson, H. et al. (1998) Scandinavian J. Immunol. 48:116-23.

1. A pharmaceutical composition comprising isolated non-recombinant mannose binding lectin (MBL) substantially free from activated MBL associated serine proteases (MASPs) together with a pharmaceutically acceptable carrier or diluent.

2. A composition according to claim 1, wherein the MBL is substantially free from MASPs.

3. A composition according to claim 1 or claims 2, wherein the MBL is human MBL.

4. A composition according to any one of claims 1 to 3, wherein the MBL is obtained by a method comprising:
(i) providing a complex of non-recombinant MBL and one or more MASPs;
(ii) incubating the complex in a suitable buffer to dissociate the MBL from the one or more MASPs; and
(iii) separating the MBL from the one or more MASPs.

5. A composition according to claim 4, wherein the buffer in step (ii) is an EDTA/acetate buffer at a pH of from 4.0 to 5.0.

6. A composition according to claim 4 or claim 5, wherein the buffer in step (ii) comprises NaCl.

7. A composition according to any one of claims 4 to 6, wherein step (iii) includes a chromatographic method and/or filtration.

8. A composition of claim 7, wherein the chromatographic method is selected from the group consisting of: size exclusion chromatography and ion exchange chromatography.

9. A method of producing a pharmaceutical composition, the method comprising:
(i) providing a complex of non-recombinant MBL and one or more MASPs;
(ii) dissociating the MBL from at least some of the one or more MASPs;
(iii) separating the MBL from at least some of the one or more MASPs; and
(iv) admixing the resulting MBL from step (iii) with a pharmaceutically acceptable carrier or diluent.

10. A method of claim 9, wherein step (ii) involves incubating the complex in a suitable buffer.

11. A method according to claim 10, wherein the buffer is an EDTA/acetate buffer at a pH of from 4.0 to 5.0.

12. A method according to claim 10 or claim 11, wherein the buffer comprises NaCl.

13. A method according to any one of claims 9 to 12, wherein step (iii) includes a chromatographic method and/or filtration.

14. A method of claim 13, wherein the chromatographic method is selected from the group consisting of: size exclusion chromatography and ion exchange chromatography.

15. A method according to any one of claims 9 to 14, wherein step (i) comprises providing a side fraction from plasma fraction processes.

16. A method according to claim 15, wherein step (i) further comprises separating complexes of non-recombinant MBL and one or more MASPS from other plasma proteins present in the side fraction from plasma fraction processes by mannan affinity chromatography.

17. A pharmaceutical composition obtained by the method of any one of claims 9 to 16.

18. The pharmaceutical composition of claim 17, wherein the composition is substantially free of activated MASPs.

19. A method of treating or preventing a disease in a subject, the method comprising administering to the subject an effective amount of a pharmaceutical composition according to any one of claims 1 to 8, 17 or 18.

20. A method according to claim 19, wherein the subject is a bone marrow allograft recipient.

21. A method according to claim 19, wherein the subject is immunodeficient.

22. A method according to claim 19, wherein the subject has community acquired or nosocomial septicaemia.

23. A method according to claim 19, wherein the subject is a low birthweight and/or premature infant.

24. A method according to claim 19, wherein the subject is infected with a pathogen.

25. A method according to any one of claims 19 to 24, wherein the subject has an MBL deficiency.

26. A method according to claim 25, wherein the subject is an infant at risk from developing acute lymphoblastic leukaemia.

27. A composition comprising isolated non-recombinant MBL, said composition being substantially free of activated MASPs, for use prophylactically or in therapy.

28. A composition comprising isolated non-recombinant MBL, said composition being substantially free of MASPs, for use prophylactically or in therapy.

29. Use of a composition comprising isolated non-recombinant MBL, said composition being substantially free of MASPs, in the manufacture of a medicament for use in administering to a subject in need of said composition.

30. Use according to claim 29, wherein the subject is a bone marrow allograft recipient.

31. Use according to claim 29, wherein the subject is immunodeficient.

32. Use according to claim 29, wherein the subject has community acquired or nosocomial septicaemia.

33. Use according to claim 29, wherein the subject is an infant at risk from developing has acute lymphoblastic leukaemia.

34. Use according to claim 29, wherein the subject is a low birthweight and/or premature infant.

35. Use according to claim 29, wherein the subject is infected with a pathogen

36. Use according to any one of claims 29 to 35, wherein the composition is substantially free of MASPs.

37. A peptide of formula X-R1-Arg-R2-Y, wherein R1-Arg-R2 is a peptide consisting of 6 or more contiguous amino acids derived from the MASP cleavage site of a complement protein; X is NH₂, a blocking group or a detectable label; and Y is COOH or a detectable label, provided that when X is NH₂ or a blocking group, Y is not COOH and when Y is COOH, X is not NH₂ or a blocking group.

38. A peptide according to claim 37, wherein the complement protein is C4.

39. A peptide according to claim 38, wherein the C4 protein is human C4 and the cleavage site comprises Arg756.

40. A peptide according to any one of claims 37 to 39, wherein X is a quencher molecule and Y is a fluorescent label, or vice-versa, such that a fluorescent signal is obtained when the substrate is cleaved.

41. Use of a peptide according to any one of claims 37 to 40, in a method of determining the presence of MASP activity in a sample.

42. Use according to claim 41, wherein the sample is a composition according to any one of claims 1 to 8, 17 or 18.

43. A method of determining the presence of MASP activity in a sample which method comprises contacting the sample with a peptide according to any one of claims 37 to 40 and determining whether said peptide has been cleaved.

44. A method according to claim 43, wherein the sample is a composition according to any one of claims 1 to 8, 17 or 18.

45. A method of producing a pharmaceutical composition according to any one of claims 1 to 3 which method comprises:
(i) providing a complex of non-recombinant MBL and one or more MASPs;
(ii) incubating the complex in a suitable buffer to dissociate the MBL from the one or more MASPs;
(iii) separating the MBL from the one or more MASPs;
(iv) screening the MBL obtained from (iii) for MASP activity using a method according to claim 43; and
(v) admixing the resulting purified MBL with a pharmaceutically acceptable carrier or diluent.
